(19) United States
(12) Patent Application Publication
(10) Pub. No.: US 2026/0368899 A1
(43) Pub. Date: Nov. 19, 2026

(54) METHOD, SYSTEM AND COMPUTER PROGRAM ELEMENT FOR PROGRESSION MONITORING OF AN X-RAY IMAGE SERIES

(71) Applicant: KONINKLIJKE PHILIPS N.V., EINDHOVEN (NL)

(72) Inventors: ALEKSANDR EFITOROV, MOSKVA (RU); HEINRICH SCHULZ, HAMBURG (DE); STEFFEN RENISCH, HAMBURG (DE)

(21) Appl. No.: 19/139,553

(22) PCT Filed: Dec. 13, 2023
(86) PCT No.: PCT/EP2023/085462

§ 371 Date: Jun. 16, 2025

(30) Foreign Application Priority Data
Dec. 22, 2022 (RU) 2022133845

(51) Int. Cl. G06T 7/00; G06T 7/11; G06V 10/25

(52) U.S. Cl. CPC G06T 7/0016; G06T 7/11; G06V 10/25; G06T 2207/10016; G06T 2207/10116; G06T 2207/20104; G06T 2207/30048; G06T 2207/30061; G06V 2201/031

(57) ABSTRACT

The invention concerns a method, a system and a computer program element for progression monitoring of an X-ray image series. which comprises the steps of receiving the X-ray image series captured from a patient by an X-ray system, selecting a region of interest (ROI) from at least one image of the X-ray image series, dividing the selected ROI of each image of the plurality of X-ray images into spatial patches using a processing unit, determining a breathing cycle and a heartbeat cycle from the patches using the processing unit, eliminating the breathing cycle and the heartbeat cycle from the patches using the processing unit, comparing the patches of the ROI with a reference patch, determining deviating patches, which deviate from the reference patch, generating from the deviating patches a visualization map overlaid over the images of the X-ray image series, displaying the visualization map on an interface to the user.

### FIELD OF THE INVENTION

The invention relates to the field of monitoring temporal changes of a patient in X-ray images, i.e. monitoring a progression of a patient, more particular the invention relates to a progression monitoring of an X-ray image series, a system for progression monitoring of an X-ray image series, and a computer program element.

### BACKGROUND OF THE INVENTION

Despite of modern imaging methods a patient progression monitoring is still a challenging task for physicians. For instance, even for the widely used X-ray devices the obtained images for the same patient may differ because of the change of internal parameters and conditions of the capturing of the image (for example, changing parent meters may be voltage and current of the emitter, exposure time etc.), the position of the patient, the inhalation phase, or changing the capturing equipment because of technical reasons or hospital workload. These factors make it difficult to directly compare images taken consecutively one after another and/or taken on different days. Hence, different settings of the same device can complicate patient condition/progression monitoring during the treatment. Modern digital X-ray detectors provide the ability to generate an image in a short time, which make it possible to generate time series of X-ray images with high resolution, at a rate of up to several frames per seconds. For this time, series of X-ray images an objective and quantitative analysis would be highly desirable.

### SUMMARY OF THE INVENTION

Therefore, there exists a need for optimizing a progression monitoring of X-ray images of a patient. There exists a need for optimizing the image capturing process and a need for improving visualization of an X-ray image and/or an X-ray image series during progression monitoring such that a functional condition of the patient can be obtained.

An object of the invention is to provide an improved method, a system, and a computer program element for progression monitoring of an X-ray image series.

The object of the present invention is solved with the subject matter of the independent claims, wherein further embodiments are incorporated in the dependent claims.

It should be noted that any feature, function and/or element described in the following with reference to the method equally applies to the system, and vice versa. Accordingly, any feature, function, step and/or element described in the following with reference to one aspect of the present disclosure equally applies to any other aspect of the present disclosure.

According to a first aspect of the invention, a method for progression monitoring of an X-ray image series is described. The method comprises the steps of receiving the X-ray image series captured from a patient by an X-ray system, selecting a region of interest (ROI) from at least one image of the X-ray image series, dividing the selected ROI of each image of the plurality of X-ray images into spatial patches using a processing unit, determining a breathing cycle and a heartbeat cycle from the patches using the processing unit. The method further comprises the steps of eliminating the breathing cycle and the heartbeat cycle from the patches using the processing unit, comparing the patches of the ROI with a reference patch, determining deviating patches, which deviate from the reference patch, generating from the deviating patches a visualization map overlaid over the images of the X-ray image series, displaying the visualization map on an interface to the user.

In the context of the present invention, the term “X-ray image series” shall be understood to describe a sequence of X-ray images obtained by dynamic X-ray imaging, which describes a functional imaging that uses sequential images, which provides many advantages such as a high temporal resolution and flexibility in body positioning. An image series may be understood as an image series, wherein a plurality of images is generated during a time sequence. The image series may be an image sequence of a plurality of images generated one after another of the same object.

In the context of the present invention, the term “region of interest (ROI)” shall be understood to describe a sample within the medical image of particular interest for the medical diagnosis used by the user. For instance, a region of interest may be a boundary of a tumor, the bronchi, the lungs, the heart, the bronchial tube, the lung region, wherein this list is not limited. The kind and size of the ROI may depend on the medical image to be analyzed, the anatomical structure that could be found in the medical image, and a part of a body of the patient, which undergoes medical image analysis.

In the context of the present invention, the term “patch” shall be understood to describe a spatial region of predetermined size wherein the size may be defined by the user, may be predefined or can be defined during the image analysis. In particular, a patch shall be understood to describe at least one spatial region at the image, which is homogeneous, in other words includes similar image features. The minimum size of the patch may be at least a pixel, wherein preferably the size of the patch/patches may be larger than one pixel. The region of interest may be divided into a plurality of patches.

In other words, a method is described which allows analysis of the dynamics of X-ray images series, in particular chest X-ray images series, observing one or several breathing cycles with high temporal resolution and not only the analysis of the intensities of the single chest snapshot collected in different times. On the other hand, it may be possible to use an incomplete breathing cycle, which means not a full breathing cycle of inhalation and exhalation. It may be possible to estimate the “global homogenous” contraction or expansion and correct it. Hence, the breathing motion may be estimated using image data from less than a breathing cycle. The method comprises the selection of a region of interest (ROI) on a chest X-ray image series and afterwards a time series dynamic analysis is performed. Time series dynamic analysis may include the following steps of splitting the selected region of interest into spatial patches and an extraction of frequencies of the breathing cycle and heartbeat cycle. The patches are compared with a reference area, which is a reference patch. The extraction of the breathing cycle and the heartbeat cycle may comprise an intensity-based time series analysis, which may include the extraction of major frequencies. For performing the method, a processing unit may be used, which is able to perform one or more of the method steps. For instance, the X-ray image series may be received at the processing unit and/or at a memory accessed by the processing unit. The images may be received from an X-ray imaging system. On the other hand, the images may be received from any computer, which had received the images from an X-ray system.

The elimination of the breathing and the heartbeat cycle allows compensating drifts, which may disturb the changes in the anatomical structure in the X-ray image series, which changes are anticipated to be detected. For instance, a shifting of the positions of contrasting objects like ribs and diaphragm can be decreased or almost eliminated.

The step of determining deviating patches, which deviate from the reference patch, may comprise an automatic determination of deviating areas, i.e. patches, the frequencies of which do not correspond to breathing cycle and/or heartbeat cycle or neighboring patches.

The invention may provide the advantage that for modern machines the effective radiation dose for the patient is relatively small, so several chest X-ray images scans will not introduce a significant risk or radiation burden for the patient. From the clinical personnel perspective, there is no need to reacquire an image in the case of different equipment settings or wrong inhalation phase. Further, the physician can still select from the series of images an individual appropriate image for, for example, the canonical analysis. The more accurate patient progression monitoring will help to e.g. estimate the effectiveness of a treatment strategy and operationally make necessary changes.

According to an exemplary embodiment of the invention, the step of comparing the patches with the reference patch may comprise an extraction of amplitude and/or phase information from the X-ray image based on a real and/or an imaginary part of the complex coherency. The extraction of phase information may comprise any extraction of any image signal, in particular from any image of the image series. For example, for a given image, a pair of pixel or pairs of pixel may be selected and then the time series is build based on using that image as a reference image. The pairs of pixel may refer to parts of corresponding patches, in particular to patches, which may be tracked along the image series. Further, the extraction of phase information may be an advanced mathematical model to extract appearance changes in the image patches.

According to an exemplary embodiment of the invention, wherein determining deviating patches may comprise at least one of an independent component analysis, a computing of a statistical moment, or a maximum pixel value change rate. For instance, for this step the breathing cycle and the heartbeat cycle are removed by excluding the corresponding independent component and restoring the original image, i.e. image signal, without it. Hence, it could be any or even a combination of the above mentioned for determining deviating patches.

According to an exemplary embodiment of the invention, the region of interest may be selected automatically or manually by the user. The region of interest may be a region, which is an anatomical structure from which the progression should be/is monitored. The region of interest may be determined by the user, depending on the anatomical structure, which should be investigated. On the other hand, it may be determined automatically e.g., by an artificial intelligence, which may be trained by X-ray image data comprising different anatomical structures such that the artificial intelligence may be able to determine the respective anatomical structure shown in the X-ray image series. Additionally, the reference patch may be selected automatically or manually by the user, wherein the reference patch is a part of the X-ray image, which is determined as a normal element in the X-ray image. For instance, the reference patch may be a patch within bone, heart, or definitely healthy tissue, dependent from the task, which should be done from the X-ray image series. If an artificial intelligence is used for determining the patch, the artificial intelligence may be trained with healthy tissue data for respective anatomical structures. The reference patch as the normal element in the x-ray image could be a normal element in the sense of healthy tissue or healthy bone or normal functioning anatomical structure.

According to an exemplary embodiment of the invention, the steps of dividing the selected ROI of each image of the plurality of X-ray images, determining a breathing cycle and a heartbeat cycle from the patches, determining a breathing cycle and a heartbeat cycle from the patches, eliminating the breathing cycle and the heartbeat cycle from the patches, comparing the patches with a reference patch, determining deviating patches, which deviate from the reference patch, and generating from the deviating patches a visualization map may be performed for each image of the X-ray image series in particular, each of the above-mentioned steps may be performed for each single image of the X-ray series. In particular, each step may be performed one after another for each single image of the time series, or the steps may be carried out one after another but simultaneously for each image of the time series of images. In other words, all images of the image series may have to undergo all of the above mentioned steps, such that an estimation of dynamic parameters of the tissue can be carried out for and over the whole image series.

According to an exemplary embodiment of the invention, determining the breathing cycle and the heartbeat cycle may comprise determining the breathing cycle and the heartbeat cycle from at least one of a dynamic of the total patches intensity, or based on a pixel value change rate. In other words, the patch intensity as such may not be measured, but the corresponding patches are tracked spatially. The corresponding patches are the patches of each image of the image series. For instance, a first image comprises patch x1, x2 and x3 and a second image comprises patch y1, y2, x3, wherein the patch x1 anatomically corresponds to the patch y1 of the other image, i.e. the patches x1 and y1 depict identical anatomical structures (which may have been moved relative to other anatomical structures due to some form of motion), and so on. The corresponding patches may be described as patches of different images, which correspond in their position in the image. The spatially tracked patches may be anatomical structures (bones, tissues, boundaries, etc.). The step of determining the different cycles may be carried out by the processing unit or any computer element.

According to an exemplary embodiment of the invention, for the determination of the breathing cycle and/or of the heartbeat cycle one or more sensor may be used in addition. For instance, data of a breathing sensor (spirometer) or a heart measurement device, heart electrodes, or sensor data used for electrocardiogram could be included. This sensor signal may be used for eliminating the breathing cycle and/or the heartbeat cycle in the image(s) of the time series.

According to an exemplary embodiment of the invention, the dynamic X-ray series may be a time series of chest X-ray images. In other words, the X-ray images are images taken from a chest of a patient during an X-ray image processing wherein a sequence of X-ray images over time is generated.

According to an exemplary embodiment of the invention, the method may further comprise the steps of generating a total score for the ROI based on a ratio of normal patches and deviated patches, and comparing the total score with previous X-ray images. The score and the spatial distribution of deviated patches could be compared with previous observations, which means with previous images of a previous time series.

The method in general may comprise the assessment of the functional properties of the tissue are, which may be the patches of the ROI, in dynamic. Accordingly, at least a short time series is necessary for generating an image series from which the functional properties can be determined.

According to an exemplary embodiment of the invention, the method may further comprise the step of comparing the patches of the received X-ray images series with patches received from a further X-ray images series. Hence, the corresponding patches, which may be selected manually by the user may then be compared with patches from the previous session. For both patches, the basic frequencies like breathing and heartbeat should be eliminated from both time series, such that a comparison between the patches from the different time series can be performed.

According to an exemplary embodiment of the invention, the visualization map may be selected from at least one of breathing map, perfusion map, wherein any visualization of spatial-temporal map of the ROI could be applied. Further, an average level of synchronization may be calculated or a relative change in intensity for the selected ROI. This value(s) may be compared with a threshold, for example mean plus 3 sigma from the same area (ROI or only a patch of the ROI) or from the selected reference patch. Then for the selected area, pixels relative to this baseline may be painted. The visualization map may be chosen by the user depending on the selected region of interest and/or depending on the diagnosis of the patient. Further, the visualization map may be chosen automatically, depending on the chosen region of interest or the anatomical structure determined (by the processing unit using an artificial intelligence or any other object detection unit) in the X-ray image (series).

According to an exemplary embodiment of the invention, the generation of the visualization map may further comprise the generation of an ROI color map depending on the selected visualization map, which is displayed as the overlay over the plurality of X-ray images. In particular, the visualization map visualizes the selected region of interest and indicates this region with a determined color. Furthermore, the ROI color map may be a spatial color map, which may indicate in the region of interest the differentiating patches by colors.

According to an exemplary embodiment of the invention, the generation of the visualization map may comprise an illustration of the functional condition of the ROI. In particular, the functional condition of the anatomical structure of interest can be visualized. For instance, whether both lobes of the lung are functioning properly, i.e. inhaling and exhaling can be derived from the visualization map and the expansion of the lung during inhaling and exhaling can be derived. The same applies to other anatomical structures, like the heart beating properly or a perfusion of the lung etc.

According to an exemplary embodiment of the invention, the received X-ray images series is a time series of consecutive X-ray images. Hence, the plurality of images received during the x-ray imaging, are images received one after another, wherein a plurality of x-ray images are generated during the execution of the imaging process.

According to a second aspect of the invention, a system is described for progression monitoring of an X-ray image series. The system may comprise a processing unit configured for
receiving the X-ray image series from a patient, selecting a region of interest (ROI) from at least one image of the X-ray image series, dividing the selected ROI of each of the image of the X-ray image series into spatial patches using a processing unit, determining a breathing cycle and a heartbeat cycle from the patches using the processing unit, eliminating the breathing cycle and the heartbeat cycle from the patches using the processing unit, comparing the patches with a reference patch, determining deviating patches, which deviate from the reference patch, generating from the deviating patches a visualization map overlaid over the images of X-ray image series. Further, the system may comprise an interface, wherein the interface is configured to display to the user the visualization map generated by the processing unit.

The system may be a stand-alone unit, which can be attached and/integrated to any suitable X-ray system. On the other hand, the system may comprise an X-ray system comprising respective X-ray source and X-ray detector for performing an X-ray imaging and including the processing unit, which is configured, for performing the steps as described above.

The proposed system and also the proposed method may be in principle applicable to any hospital with X-ray machines, which can provide a rapid X-ray capturing rate. All of the required method steps and/or computations could be provided in the cloud, which may also include automatic region of interest selection (for instance based on lung segmentation) with automatically uploaded chest X-ray image data. For example, a user control and the result demonstration could be provided on the display comprised an interface of the system or it may also be provided in a tablet application.

According to a third aspect of the invention, a computer program element for progression monitoring of an X-ray image series. The computer program element, when being executed by a processing unit of a system may be adapted to cause the system to receive the X-ray image series captured from a patient, select a region of interest (ROI) from at least one image of the X-ray images series, divide the selected ROI of each of image of the X-ray image series into spatial patches using a processing unit, determine a breathing cycle and a heartbeat cycle from the patches using the processing unit, eliminate the breathing cycle and the heartbeat cycle form the patches using the processing unit, compare the patches with a reference patch, determine deviating patches, which deviate from the reference patch, generate from the deviating patches a visualization map overlaid over the images of the X-ray image series, and to display the visualization map on an interface to the user.

The computer program element may be part of a computer program, but it can also be an entire program by itself. For example, the computer program element may be used to update an already existing computer program to get to the present invention.

The program element may be stored on a computer readable medium. The computer readable medium may be seen as a storage medium, such as for example, a USB stick, a CD, a DVD, a data storage device, a hard disk, or any other medium on which a program element as described above can be stored.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

It has to be noted that embodiments of the invention have been described with reference to different subject matters. In particular, some embodiments have been described with reference to apparatus/system type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered as to be disclosed with this application.

### BRIEF DESCRIPTION OF THE DRAWINGS

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

FIG. 1 illustrates a flow diagram of a method according to an embodiment of the invention.

FIG. 2 illustrates different images of an image series according to an embodiment of the invention.

FIG. 3 illustrates a determination of deviating patches according to an embodiment of the invention.

FIG. 4 illustrates a generating of a visualization map according to an embodiment of the invention.

### DETAILED DESCRIPTION OF EMBODIMENTS

The illustrations in the drawings are schematic. It is noted that in different figures similar or identical elements are provided with the same reference signs.

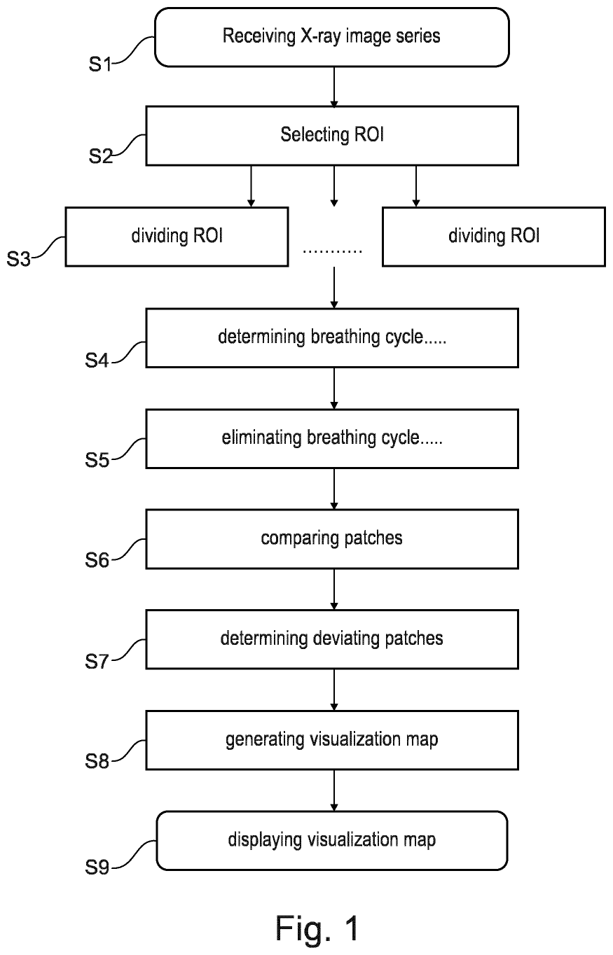

FIG. 1 illustrates a flow diagram comprising method steps according to an embodiment of the invention. The method for progression monitoring of an X-ray image series, the method comprising the steps of S1 to S9, wherein more methods steps may also be applied. The order of the steps as mentioned is only exemplary and the order is not limited to the order as described. For instance, the order of one or more steps may be changed with each other, or one or more steps may be repeated before another step is performed. The method comprises the step S1 receiving the X-ray image series captured from a patient by an X-ray system, step S2 selecting a region of interest (ROI) from at least one image of the X-ray image series, step S3 dividing the selected ROI of each image of the plurality of X-ray images into spatial patches using a processing unit. Step S3 may be comprised of further sub steps, wherein for each image of the image series the ROI is divided into patches. On the other hand, in step S3 for each image of the image series the ROI is divided into the patches simultaneously. Also the other described method steps may be carried out one after another for each image of the image series or may be carried out simultaneously for each image of the image series. Further, the method comprises step S4 determining a breathing cycle and a heartbeat cycle from the patches using the processing unit, step S5 eliminating the breathing cycle and the heartbeat cycle from the patches using the processing unit, step S6 comparing the patches of the ROI with a reference patch, step S7 determining deviating patches, which deviate from the reference patch, step S8 generating from the deviating patches a visualization map overlaid over the images of the X-ray image series, and step S9 displaying the visualization map on an interface to the user. The displaying of the visualization map may be displayed on an interface to the user, or on a display of the interface, on a display of an X-ray device, or an X-ray system. Alternatively, the displaying may be device, like

The step S6 may further comprise comparing the patches with the reference patch comprising an extraction of amplitude and/or phase information from the X-ray image based on a real and/or an imaginary part of the complex coherency respectively, which could be computed as a correlation function between spectral components of the two time series for given set of frequencies, wherein this may be carried out in a sub step of step S6. Further, the step S7 may comprise at least one of an independent component analysis, a computing of a statistical moment, or a maximum pixel value change rate, wherein each of these methods may be carried out independently from another as a sub step of step S7, or may be an extra step after step S7.

The steps S3 to S8 are performed for each image of the X-ray image series one after another or simultaneously for each image of the image series.

The method may further comprise additional steps, for example generating a total score for the ROI based on a ratio of normal patches and deviated patches, and comparing the total score with previous X-ray images. Accordingly, the present X-ray image series may be compared with another X-ray image series, for instance a historical image series, for analyzing a change in the condition of the patient or a progression of the condition of the functional properties of the analyzed anatomical structure.

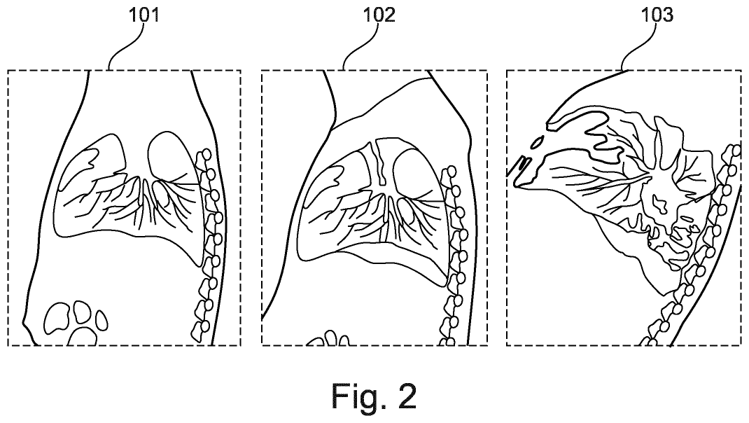

FIG. 2 shows three different images 101 to 103 of a chest of a patient. Examples of series of lateral chest X-ray images observed for the patient during the treatment are illustrated. Different positions of the patient and capturing settings of the same device may complicate patient condition monitoring, wherein when applying the method as described above, these complications can be overcome, because for each image the region of interest can be determined and can be analyzed using the method as described herein.

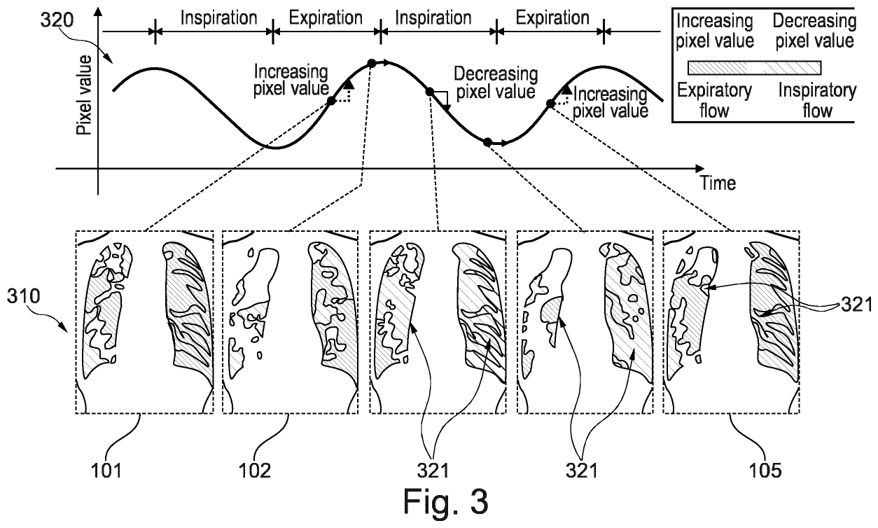

FIG. 3 shows a determination of deviating patches according to an embodiment of the invention and the illustration of a visualization map 310. The region of interest 321 is selected manually by the user, the physician, or automatically. In FIG. 3, the region of interest 321 is the lung, wherein both lobes of the lung are indicated as the region of interest 321. As can be seen at least five X-ray images 101-105 are displayed from the X-ray image series. Hence, one could say the image series of FIG. 3 comprises of at least five chest X-ray images 101 to 105. For each image 101 to 105, the region of interest 321 is indicated. Further, in each image 101 to 105 the region of interest 321 is divided into patches. Starting with image 101 the right lobe of the lung is completely indicated in the region of interest. In contrast thereto, the left lobe of the lung is not fully visible. When comparing the images 101 to 105 differences between the indicated region of interest, and thus between the patches of the left and right lobe of the lung can be compared. Further, FIG. 3 illustrates a breathing cycle 320 determined from the patches of the region of interest 321, wherein the breathing cycle 320 is determined from/over all images of the image series 101 to 105. The breathing cycle 320 is illustrated as a frequency plot and is generated for the selected patch. The breathing cycle 320 can be divided into inspiration parts and expiration parts, wherein a determination of the pixel value change rate can be performed for determining deviating patches in the region of interest. For instance, an increasing pixel value can be determined during the expiration and a decreasing pixel value can be determined for inspiration. Hence, the functional condition of the lung can be illustrated via the visualization map 310, this can be displayed to the user via a display on an interface, and the functional condition of the patient observed over the time series can be shown. The illustrated chest X-ray images may also be available for the user for a standalone observation/ analysis.

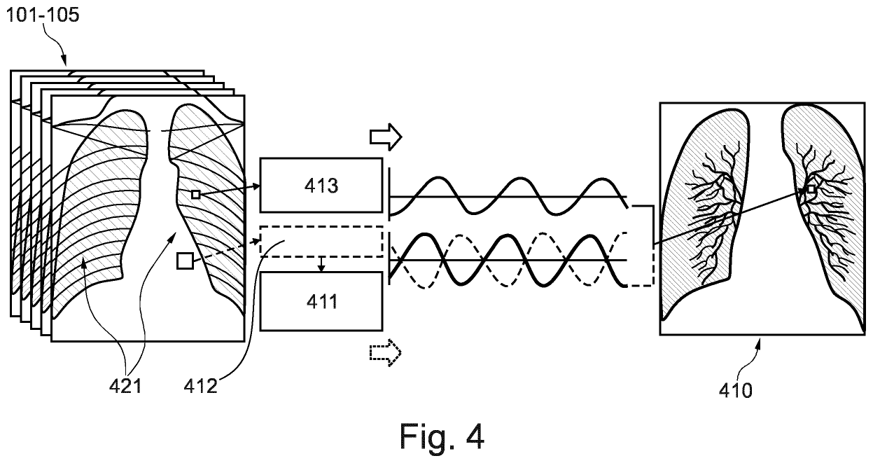

FIG. 4 shows a generating of a visualization map 410 according to an embodiment of the invention. In particular as a visualization map 410 a perfusion map generation process is shown, which is based on the dynamic X-ray time series of the images 101 to 105 of the lung. From the time series of X-ray images 101 to 105 the lung signal 413, which may be the breathing cycle and the heart signal 412, i.e. the heartbeat cycle can be determined. Afterwards these signals can be eliminated, for instance an inverted heart signal 411 can be applied for extinguishing the heart signal 412. Additionally, it may be possible to apply filters, like a high-pass or low-pass filter depending on the signal to be eliminated and the signal, which should be obtained.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. It should be noted that the term “comprising” does not exclude other elements or steps and “a” or “an” does not exclude a plurality. Also, elements described in association with different embodiments may be combined. It should also be noted that reference signs in the claims should not be construed as limiting the scope of the claims

### LIST OF REFERENCE SIGNS

S1 to S9 steps of the method
101, 102, 103 X-ray image
105 X-ray image
310 visualization map
320 breathing cycle
321 region of interest
410 visualization map
411 inverted heart signal
412 heart signal
413 lung signal
421 region of interest

1. A computer-implemented method for progression monitoring of an X-ray image series, the method comprising:
receiving the X-ray image series captured from a patient by an X-ray system;
selecting a region of interest (ROI) from at least one image of the X-ray image series;
dividing the selected ROI of each image of the plurality of X-ray images into spatial patches;
determining a breathing cycle and a heartbeat cycle from the patches;
eliminating the breathing cycle and the heartbeat cycle from the patches;
comparing the patches of the ROI with a reference patch;
determining deviating patches, which deviate from the reference patch;
generating from the deviating patches a visualization map overlaid over the images of the X-ray image series; and,
displaying the visualization map.

2. The method according to claim 1, wherein comparing the patches with the reference patch comprises extracting amplitude and/or phase information from the X-ray image based on a real and/or an imaginary part of the complex coherency, respectively.

3. The method of claim 1, wherein determining deviating patches comprises at least one of: an independent component analysis, a computing of a statistical moment, and a maximum pixel value change rate.

4. The method according to claim 1,
wherein the ROI is selected automatically or manually by the user, and/or
wherein the reference patch is selected automatically or manually by the user,
wherein the reference patch is a part of the X-ray image, which is determined as a normal element in the X-ray image.

5. The method according to claim 1, wherein dividing the selected ROI of each image of the plurality of X-ray images, determining a breathing cycle and a heartbeat cycle from the patches, determining a breathing cycle and a heartbeat cycle from the patches, eliminating the breathing cycle and the heartbeat cycle from the patches, comparing the patches with a reference patch, determining deviating patches, which deviate from the reference patch, and generating from the deviating patches a visualization map are performed for each image of the X-ray image series.

6. The method according to claim 1, wherein determining the breathing cycle and the heartbeat cycle comprises determining the breathing cycle and the heartbeat cycle from at least one of a dynamic of the total patches intensity, or based on a pixel value change rate.

7. The method according to claim 1, wherein the dynamic X-ray series is a time series of chest X-ray images.

8. The method according to claim 1, wherein the method further comprises:
generating a total score for the ROI based on a ratio of normal patches and deviated patches; and
comparing the total score with previous X-ray images.

9. The method according to claim 1, wherein the method further comprises
comparing the patches of the received X-ray images series with patches received from a further X-ray images series.

10. The method according to claim 1, wherein the visualization map is selected from at least one of a breathing map, and a perfusion map.

11. The method according to claim 1, wherein the generation of the visualization map further comprise a generating of an ROI color map depending on the selected visualization map, which is displayed as the overlay over the plurality of X-ray images.

12. The method according to claim 1, wherein generating the visualization map comprises an illustration of the functional condition of the ROI.

13. The method according to claim 1, wherein the received X-ray images series is a time series of consecutive X-ray images.

14. A system for progression monitoring of an X-ray image series, comprising:
a memory that stores a plurality of instructions;
a processor coupled to the memory and configured to execute the plurality of instructions to:
receive the X-ray image series from a patient;
select a region of interest (ROI) from at least one image of the X-ray image series;
divide the selected ROI of each of the image of the X-ray image series into spatial patches;
determine a breathing cycle and a heartbeat cycle from the patches;
eliminate the breathing cycle and the heartbeat cycle from the patches;
compare the patches with a reference patch;
determine deviating patches, which deviate from the reference patch;
generate from the deviating patches a visualization map overlaid over the images of X-ray image series;
an interface configured to display the visualization map.

15. (canceled)

16. A non-transitory computer readable medium comprising executable instructions which, when executed by at least one processor, cause the at least one processor to perform a method for progression monitoring of an X-ray image series, the method comprising:
receiving the X-ray image series captured from a patient by an X-ray system;
selecting a region of interest (ROI) from at least one image of the X-ray image series;
dividing the selected ROI of each image of the plurality of X-ray images into spatial patches;
determining a breathing cycle and a heartbeat cycle from the patches;
eliminating the breathing cycle and the heartbeat cycle from the patches;
comparing the patches of the ROI with a reference patch;
determining deviating patches, which deviate from the reference patch;
generating from the deviating patches a visualization map overlaid over the images of the X-ray image series; and
displaying the visualization map.
